(19) United States
(12) Patent Application Publication
(10) Pub. No.: US 2011/0086920 A1
(43) Pub. Date: Apr. 14, 2011

(54) Gamma Glutamylcysteine Treatment or Prevention of Oxidative Injury

(76) Inventors: Marshall Summar, Nashville, TN (US); Gary Cunningham, Nashville, TN (US); Michael Aschner, Nashville, TN (US); Truc Le, Nashville, TN (US); Rick Barr, Nashville, TN (US); Paul Moore, Nashville, TN (US)

(21) Appl. No.: 12/747,786

(22) PCT Filed: Dec. 11, 2008
(86) PCT No.: PCT/US08/86379

§ 371 Date: Nov. 2, 2010

Related U.S. Application Data
(60) Provisional application No. 61/013,576, filed on Dec. 13, 2007.

(51) Int. Cl. A61K 31/198; C12N 5/071; C12N 5/077; A61P 39/06

(52) U.S. Cl. 514/562; 435/375

(57) ABSTRACT

The present invention concerns the γ-glutamylcysteine treatment to reduce oxidative damage caused by ischemia/reperfusion events such as pulmonary hypertension, transplant, cardiac bypass and other surgeries, and trauma. In specific embodiments, preservation methods and apparatuses for preserving tissue for transplantation purposes is provided.

The present application claims benefit of priority to U.S. Provisional Application Ser. No. 60/013,576, filed Dec. 13, 2007, the entire contents of which are hereby incorporated by reference.

### BACKGROUND OF THE INVENTION

1. Field of the Invention

The present invention relates generally to the fields medicine and cell biology. More particularly, it concerns methods for reducing oxidative acute ischemia and reperfusion (ischemia/reperfusion) injury in tissues, organs and organisms. In particular, the methods address ischemia in the setting of hypoxia, stroke, heart attack, birth hypoxia, trauma, cardiac arrest, and other clinical entities involving acute ischemia/hypoxia. It also addresses ischemia/reperfusion in the settings of organ transplantation, cardiac bypass and other surgeries, pulmonary hypertension and systemic shock. The methods involve the use of γ-glutamylcysteine.

2. Description of Related Art

Free radical production and oxidative injury have been implicated in numerous disease processes. For example, pulmonary hypertension can be caused by damage to the pulmonary endothelium during cardiac bypass, which is thought to occur after reperfusion injury and oxidative stress induced apoptosis. Glutathione is a scavenger of free radicals and is important in reducing this oxidative injury to cells. It has been shown that intracellular levels of glutathione are crucial to a cell's viability (Chiba et al., Eur J Immunol 26(5):1164-1169). At present, however, there are no known methods that utilize the metabolic pathway that produces glutathione as an end-product to protect cells from oxidative damage.

### SUMMARY OF THE INVENTION

Thus, in accordance with the present invention, there is provided a method of ameliorating or preventing oxidative injury to a cell comprising contacting said cell with a composition comprising γ-glutamylcysteine (GGC). The cell may be an endothelial cell, a brain cell such as a neuronal cell or an astrocyte cell, a muscle cell, a gastrointestinal tract cell, a cardiac cell, a lung cell, a liver cell, a kidney cell, a skin cell, or eukaryotic cell of any type. GGC may be provided to said cell at between about 10 and about 5000 μm, or more particularly, at 10 μm, 25 μm, 50 μm, 100 μm, 150, μm, 200 μm, 250 μm, 300 μm, 350 μm, 400 μm, 450 μm, 500 μm, 600 μm, 700 μm, 800 μm, 900 μm, 1000 μm, 1500 μm, 2000 μm, 2500 μm, 3000 μm, 3500 μm, 4000 μm, 4500 μm, and 5000 μm. The method may further comprise contacting said cell with a free radical scavenger distinct from GGC.

In another embodiment, there is provided a method of protecting a subject from oxidative injury comprising administering to said subject a pharmaceutical composition comprising γ-glutamylcysteine (GGC). The subject may be a human, a dog, a cat, a pig, a horse, a cow, a rat, a mouse, a rabbit, a sheep, a goat, or a non-human primate. The composition may be administered via intravenous, intra-arterial, subcutaneous, intramuscular, intraspinal, dermal, intradermal, topical, or intracardiac injection, oral delivery or via inhalation. The subject has or may be suffering from a stroke, a heart attack, pulmonary hypertension, hypoxia, chemical or toxin poisoning, trauma, or reperfusion injury, or may be at risk of suffering from a stroke, a heart attack, hypoxia, chemical or toxin poisoning, pulmonary hypertension or reperfusion injury. The composition may be provided to said subject about 1, 2, 3, 4, 6, 8, 10 of 12 hours prior to or following the suffered event or the risk-inducing event. The method may further comprise administering said composition a second time. The composition may comprise GGC at between about 10 and about 5000 μm, or more particularly at 10 μm, 25 μm, 50 μm, 100 μm, 150 μm, 200 μm, 250 μm, 300 μm, 350 μm, 400 μm, 450 μm, 500 μm, 600 μm, 700 μm, 800 μm, 900 μm, 1000 μm, 1500 μm, 2000 μm, 2500 μm, 3000 μm, 3500 μm, 4000 μm, 4500 μm, and 5000 μm. The method may further comprise administering to said subject a free radical scavenger distinct from GGC.

The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the standard deviation of error for the device or method being employed to determine the value. Following long-standing patent law, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-F: Effects of γ-glutamylcysteine on Neurons and Astrocytes. Protection of cells as measured by MTT (3-(4,5-Dimethylthiazol-2-yl)-2,5-diphenyltetrazolium bromide) assay, GSH (glutathione) assay, and LDH (lactate dehydrogenase) assay.

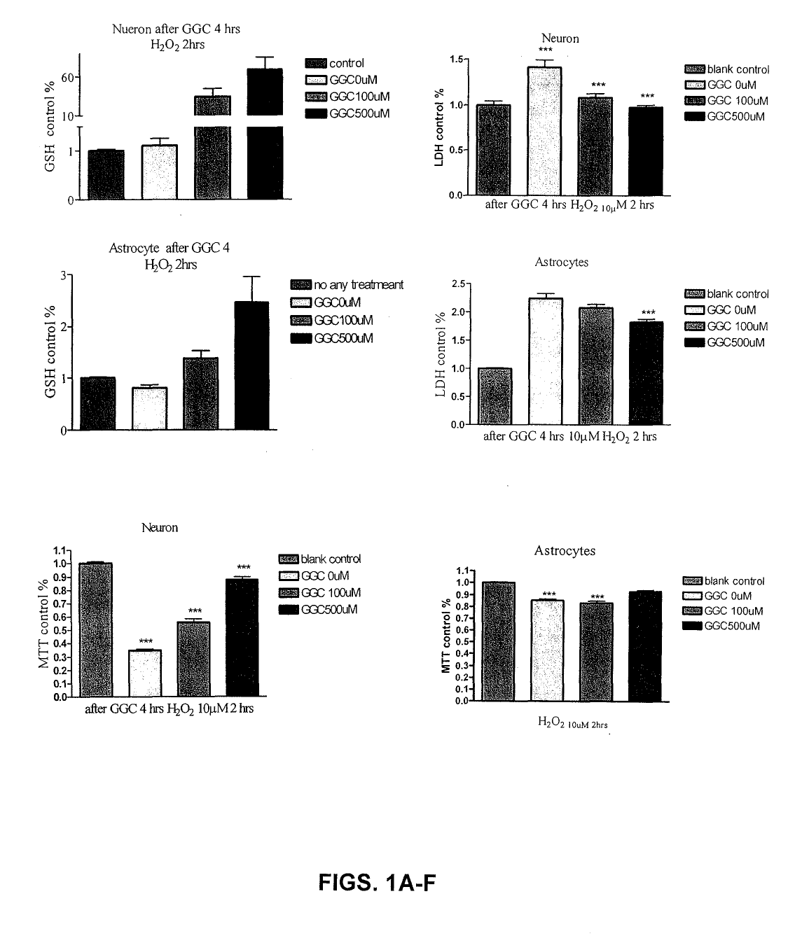

FIG. 2: IV γ-glutamylcysteine Supplementation in Rats. Two rats were administered γ-glutamylcysteine intravenously. Prior to sacrificing the animals, there were no clinically visible signs of toxicity in these animals at this dose. Plasma was collected at 0, 15, 30, 60, 90, and 120 minutes post-administration and assayed for γ-glutamylcysteine levels.

FIG. 3: Glutathione synthesis.

### DESCRIPTION OF ILLUSTRATIVE EMBODIMENTS

### I. ISCHEMIA AND REPERFUSION INJURY

Reperfusion injury refers to damage to tissue caused when blood supply returns to the tissue after a period of ischemia. The absence of oxygen and nutrients from blood creates a condition in which the restoration of circulation results in inflammation and oxidative damage through the induction of oxidative stress rather than restoration of normal function.

The damage of reperfusion injury is due in part to the inflammatory response of damaged tissues. White blood cells carried to the area by the newly returning blood release a host of inflammatory factors such as interleukins as well as free radicals in response to tissue damage. The restored blood flow reintroduces oxygen within cells that damages cellular proteins, DNA, and the plasma membrane. Damage to the cell's membrane may in turn cause the release of more free radicals. Such reactive species may also act indirectly in redox signaling to turn on apoptosis. Leukocytes may also build up in small capillaries, obstructing them and leading to more ischemia.

Reperfusion injury plays a part in the brain's ischemic cascade, which is involved in stroke and brain trauma. Repeated bouts of ischemia and reperfusion injury also are thought to be a factor leading to the formation and failure to heal of chronic wounds such as pressure sores and diabetic foot ulcers. Continuous pressure limits blood supply and causes ischemia, and the inflammation occurs during reperfusion. As this process is repeated, it eventually damages tissue enough to cause a wound.

In prolonged ischemia (60 minutes or more), hypoxanthine is formed as breakdown product of ATP metabolism. The enzyme xanthine dehydrogenase is converted to xanthine oxidase as a result of the higher availability of oxygen. This oxidation results in molecular oxygen being converted into highly reactive superoxide and hydroxyl radicals. Xanthine oxidase also produces uric acid, which may act as both a prooxidant and as a scavenger of reactive species such as peroxinitrite. Excessive nitric oxide produced during reperfusion reacts with superoxide to produce the potent reactive species peroxynitrite. Such radicals and reactive oxygen species attack cell membrane lipids, proteins, and glycosaminoglycans, causing further damage. They may also initiate specific biological processes by redox signaling.

Oxidative injury stems from transplant surgery, and this presents a key limiting factor in the success of organ transplantation, as well as the treatment of surgical injury and systemic shock. The lack of methods to minimize ischemia/reperfusion damage caused by the absence of blood flow or oxygen to the affected tissues or organs is only part if the problem, as restarting blood flow after more than about ten minutes of ischemia is typically more damaging than the ischemia itself.

The inventors now propose that insufficient levels of glutathione may contribute to oxidative damage in significant oxidative stress. Supportive of this hypothesis, an insertion/deletion polymorphism in the gene for the glutamate cysteine ligase catalytic subunit (GCLC) has been associated with the development of postoperative pulmonary hypertension in children following bypass (Willis et al., Free Radic Biol Med 34:72-76). Exogenous γ-glutamylcysteine would bypass this rate-limiting enzyme and possibly increase the production of glutathione available to scavenge free radicals and decrease cellular oxidative injury. The inventors have shown that γ-glutamylcysteine can been significantly reduce cellular death in response to an oxidative stress in vitro. Thus, they propose the use of this compound for the treatment/prevention of various forms of oxidative damage, as explained further, below.

### II. γ-GLUTAMYLCYSTEINE

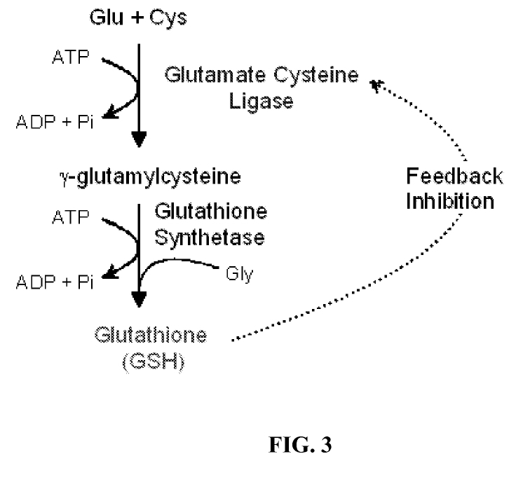

Glutathione is synthesized in two steps, the first of which combines glutamate and cysteine to form γ-glutamylcysteine using the enzyme glutamate cysteine ligase, a rate-limiting enzyme. Next, glycine is added to the C-terminus of γ-glutamylcysteine via the enzyme glutathione synthetase. FIG. 3. As discussed above, glutathione is a scavenger of free radicals and is important in reducing this oxidative injury to cells. Thus, the inventors propose to provide γ-glutamylcysteine as a way of increasing glutathione content in cells.

### III. PULMONARY HYPERTENSION

In medicine, pulmonary hypertension (PH) is an increase in blood pressure in the pulmonary artery, pulmonary vein, or pulmonary capillaries, together known as the lung vasculature, leading to shortness of breath, dizziness, fainting, and other symptoms, all of which are exacerbated by exertion. Depending on the cause, pulmonary hypertension can be a severe disease with a markedly decreased exercise tolerance and right-sided heart failure. It can be one of five different types: arterial, venous, hypoxic, thromboembolic, or miscellaneous.

The most common cause of pulmonary hypertension is left heart failure leading to pulmonary venous hypertension. This may be due to systolic or diastolic malfunction of the left ventricle or due to valvular dysfunction such as mitral regurgitation, mitral stenosis, aortic stenosis, or aortic regurgitation. It usually manifests as pulmonary edema or pleural effusions. Common causes of pulmonary arterial hypertension include HIV, scleroderma and other autoimmune disorders, cirrhosis and portal hypertension, sickle cell disease, congenital heart disease, and others. Use of weight loss pills such as Fen-Phen, Aminorex, fenfluramine (Pondimin), and phentermine led to the development of PAH in the past.

Human herpesvirus 8, also associated with Kaposi's sarcoma, has been demonstrated in patients with PAH, suggesting that this virus may play a role in its development. Recent studies have been unable to find an association between human herpesvirus 8 and idiopathic pulmonary arterial hypertension.

When a family history exists, the disease is termed familial pulmonary arterial hypertension (FPAH). IPAH and FPAH are now considered to be genetic disorders linked to mutations in the BMPR2 gene, which encodes a receptor for bone morphogenetic proteins, as well as the 5-HT(2B) gene, which codes for a serotonin receptor. There seems to be an association of idiopathic PAH (not only PAH caused by heart malformations) and Trisomy 21.

Pulmonary embolism also leads to pulmonary hypertension, acutely as well as chronically. Treatments for these two conditions are vastly different. Schistosomiasis is a very common cause of pulmonary hypertension in endemic areas such as the Nile river due to obstruction of pulmonary vessels with the parasite.

Lung diseases that lower oxygen in the blood (hypoxia) are well known causes of pulmonary hypertension (WHO Group III), including COPD, interstitial lung disease such as IPF, Pickwickian syndrome or obesity-hypoventilation syndrome, and possibly sleep apnea. Other causes include sarcoidosis, histiocytosis X, and fibrosing mediastinitis (WHO Group V). When none of these causes can be found, the disease is termed idiopathic pulmonary arterial hypertension (IPAH). There appears to be a link between IPAH and thyroid diseases, but this is not regarded as causative.

Whatever the initial cause, pulmonary arterial hypertension involves the vasoconstriction or tightening of blood vessels connected to and within the lungs. This makes it harder for the heart to pump blood through the lungs, much as it is harder to make water flow through a narrow pipe as opposed to a wide one. Over time, the affected blood vessels become both stiffer and thicker, in a process known as fibrosis. This further increases the blood pressure within the lungs and impairs their blood flow. In addition, the increased workload of the heart causes thickening and enlargement of the right ventricle, making the heart less able to pump blood through the lungs, causing right heart failure. As the blood flowing through the lungs decreases, the left side of the heart receives less blood. This blood may also carry less oxygen than normal. Therefore it becomes harder and harder for the left side of the heart to pump to supply sufficient oxygen to the rest of the body, especially during physical activity.

Pathogenesis in pulmonary venous hypertension is completely different. There is no obstruction to blood flow in the lungs. Instead, the left heart fails to pumps blood efficiently, leading to pooling of blood in the lungs. This causes pulmonary edema and pleural effusions. In hypoxic pulmonary hypertension, the low levels of oxygen are thought to cause vasoconstriction or tightening of pulmonary arteries. This leads to a similar pathophysiology as pulmonary arterial hypertension. In chronic thromboembolic pulmonary hypertension, the blood vessels are blocked or narrowed with blood clots. Again, this leads to a similar pathophysiology as pulmonary arterial hypertension.

The inventor propose the use of γ-glutamylcysteine in a method of treating the oxidative damage ensuing from pulmonary hypertension, in particular that which ensues from cardiac bypass surgery and pediatric settings.

### IV. TRANSPLANT APPLICATIONS

The present inventors also propose that γ-glutamylcysteine can be used to prevent damage to tissues and organs flowing from ischemic events in transplant surgery. The invention described herein thus comprises the use of γ-glutamylcysteine to treat, preserve and protect tissues that will be or have been subjected to transplant-related ischemia/reperfusion.

In embodiments of the invention, an organism, organ or tissue can be exposed to γ-glutamylcysteine for about, at least about, or at most about 30 seconds, 1, 2, 3, 4, 5, 10, 15, 20, 25, 30, 35, 40, 45, 50, 55 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days, or 1, 2, 3, 4, 5 or 6 weeks, and any combination or range derivable therein.

In particular embodiments, it is contemplated that an organ will be treated prior to, during, and/or after transplant. In such cases, the exposure may be conducted relative to the time of organ removal or to implantation. Again, the exposure may be initiated 30 seconds, 1, 2, 3, 4, 5, 10, 15, 20, 25, 30, 35, 40, 45, 50, 55 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days, or 1, 2, 3, 4, 5 or 6 weeks prior to removal and/or subsequent to transplant.

A. Transplanted Tissue and Organs

Various tissues and organs are contemplated as being subject to transplantation according to the present invention. In particular, the tissue may be derived from skin, bone, bone marrow, cartilage, cornea, skeletal muscle, cardiac muscle, cardiac valve, smooth muscle, blood vessels, a limb or a digit, including parts or all of the aforementioned structures. Organs include kidney, pancreas, liver, heart, lung, bowel or portions thereof. Organs for transplants may be monitored to assess their condition, particularly with respect to use as a transplant. Such methods are described in U.S. Pat. No. 5,699,793.

B. Other Preservation Agents

A variety of preservation solutions have been disclosed in which a tissue or organ is surrounded or perfused with the preservation solution for storage. One of the most commonly used solutions is ViaSpan® (Belzer UW), which employed with cold storage. Other examples of such solutions or components of such solutions include the St. Thomas solution (Ledingham et al., 1987), Broussais solution, UW solution (Ledingham et al., 1990), Celsior solution (Menasche et al., 1994), Stanford University solution, and solution B20 (Bernard et al., 1985), as well as those described and/or claimed in U.S. Pat. Nos. 6,524,785; 6,492,103; 6,365,338; 6,054,261; 5,719,174; 5,693,462; 5,599,659; 5,552,267; 5,405,742; 5,370,989; 5,066,578; 4,938,961; and, 4,798,824.

Allopurinol inhibits xanthine oxidase, blocking the conversion of xanthine and oxygen to superoxide and uric acid. Glutathione is used as an antioxidant with membrane-stabilizing properties. Dexamethasone also stabilizes membranes. Magnesium seems to counteract some of the effects of intracellular calcium and the sulfate ion resists cell swelling because it is relatively impermeable to cell membranes.

In addition to solutions, other types of materials are also known for use in transporting organs and tissue. These include gelatinous or other semi-solid material, such as those described, for example, in U.S. Pat. No. 5,736,397.

Some of the systems and solutions for organ preservation specifically involve oxygen perfusion in the solution or system to expose the organ to oxygen because it is believed that maintaining the organ or tissue in an oxygenated environment improves viability. Kuroda et al., 1988 and U.S. Pat. Nos. 6,490,880; 6,046,046; 5,476,763; 5,285,657; 3,995,444; 3,881,990; and, 3,777,507. Isolated hearts that are deprived of oxygen for more than four hours are believed to lose vigor and not be useful in the recipient because of ischemic/reperfusion injury. U.S. Pat. No. 6,054,261.

Moreover, many, if not all, of the solutions and containers for organ preservation and transplantation involve hypothermia (temperature below room temperature, often near but not below 0° C.), which has been called the “bed rock of all useful methods of organ and tissue preservation.” U.S. Pat. No. 6,492,103.

As discussed in U.S. Pat. Nos. 5,952,168, 5,217,860, 4,559,258 and 6,187,529 (incorporated specifically by reference), biological materials can be preserved, for example, for keeping transplantable or replaceable organs long-term. Cells, tissue/organs, or cadavers can be provided with compounds that enhance or maintain the condition of organs for transplantation. Such methods and compositions include those described in U.S. Pat. Nos. 5,752,929 and 5,395,314.

It is contemplated that any agent or solution used with a biological sample that is living and that will be used as a living material will be pharmaceutically acceptable or pharmacologically acceptable. The phrase “pharmaceutically-acceptable” or “pharmacologically-acceptable” refers to molecular entities and compositions that do not produce an allergic or similar untoward reaction when administered to a human. The preparation of an aqueous composition that contains a protein as an active ingredient is well understood in the art. Typically, such compositions are prepared either as liquid solutions or suspensions; solid forms suitable for solution in, or suspension in, liquid prior to use can also be prepared. Any of the aforementioned solutions and transport strategies are considered suitable vehicles to carry γ-glutamylcysteine to the tissue or organ in question

A number of drugs can be administered to a patient after receiving an organ transplant to assist in the recovery process. Such drugs include compounds and agents that reduce or inhibit an immune response against the donated organ.

C. Preservation Apparati

Systems or containers for transporting organs and tissues have also been developed through the years. Any of these embodiments may be combined with apparati of the invention, which allow for use with oxygen antagonists.

Most involve cooling systems for implementation, for example, those described in U.S. Pat. Nos. 4,292,817, 4,473,637, and 4,745,759, which employ active refrigeration with a cooling liquid that is pumped through the system. Several sophisticated devices have been designed involving multiple chambers or dual containers, such as is U.S. Pat. Nos. 5,434,045 and 4,723,974.

Some constitute a system in which an apparatus is devised for perfusion of the organ or tissue in a preservation solution, as is described in U.S. Pat. Nos. 6,490,880; 6,100,082; 6,046,046; 5,326,706; 5,285,657; 5,157,930; 4,951,482; 4,502,295; and 4,186,565.

Some of the systems and solutions for organ preservation specifically involve oxygen perfusion in the solution or system to expose the organ to oxygen because it is believed that maintaining the organ or tissue in an oxygenated environment improves viability. Kuroda et al., 1988; U.S. Pat. Nos. 6,490,880; 6,046,046; 5,476,763; 5,285,657; 3,995,444; 3,881,990; and 3,777,507. Isolated hearts that are deprived of oxygen for more than four hours are believed to lose vigor and not be useful in the recipient because of ischemic/reperfusion injury. U.S. Pat. No. 6,054,261.

### V. TRAUMA, SURGERY AND SYSTEMIC SHOCK

A. Trauma and Hemorrhage

Physical trauma is a serious and body-altering physical injury, such as the removal of a limb. Blunt force trauma, a type of physical trauma caused by impact or other force applied from or with a blunt object, whereas penetrating trauma is a type of physical trauma in which the skin or tissues are pierced by an object. Trauma can also be described as both unplanned, such as an accident, or planned, in the case of surgery. Both can be characterized by mild to severe tissue damage, blood loss and/or shock, and both may lead to subsequent infection, including sepsis. The present invention provides to treatment of trauma, including both pre-treatment (in the case of a medical procedure) and treatment after trauma injury as occurred.

Traumatic hemorrhage accounts for much of the wide ranging international impact of injury, causing a large proportion of deaths and creating great morbidity in the injured. Despite differences in pre-hospital care, the acute management of traumatic hemorrhage is similar around the world and follows well accepted published guidelines. A critically injured patient's care occurs as four, often overlapping segments: the resuscitative, operative, and critical care phases. The diagnosis and control of bleeding should be a high priority during all of the phases of trauma care and is especially important in the patient who is in hemorrhagic shock. Early attempts at hemorrhage control include direct control of visible sources of severe bleeding with direct pressure, pressure dressings, or tourniquets; stabilization of long bone and pelvic fractures; and keeping the patient warm. During the resuscitative phase, warmed intravenous fluids, hypotensive resuscitation prior to surgical control of hemorrhage, and appropriate transfusion of blood and blood products are provided. In the operative phase, surgical control of the hemorrhage and any other injury, and additional transfusion is provide. Finally, the critical care phase provides for post-operative support and tissue perfusion.

B. Surgery

Surgery uses operative manual and instrumental techniques on a patient to investigate and/or treat a pathological condition such as disease or injury, to help improve bodily function or appearance, or sometimes for some other reason. The present invention can address trauma resulting from surgeries, as defined further below.

As a general rule, a procedure is considered surgical when it involves cutting of a patient's tissues or closure of a previously sustained wound. Other procedures that do not necessarily fall under this rubric, such as angioplasty or endoscopy, may be considered surgery if they involve common surgical procedure or settings, such as use of a sterile environment, anesthesia, antiseptic conditions, typical surgical instruments, and suturing or stapling. All forms of surgery are considered invasive procedures; so-called noninvasive surgery usually refers to an excision that does not penetrate the structure being addressed (e.g., laser ablation of the cornea) or to a radiosurgical procedure (e.g., irradiation of a tumor). Surgery can last from minutes to hours.

Surgical procedures are commonly categorized by urgency, type of procedure, body system involved, degree of invasiveness, and special instrumentation. Elective surgery is done to correct a non-life-threatening condition, and is carried out at the patient's request, subject to the surgeon's and the surgical facility's availability. Emergency surgery is surgery which must be done quickly to save life, limb, or functional capacity. Exploratory surgery is performed to aid or confirm a diagnosis. Therapeutic surgery treats a previously diagnosed condition.

Amputation involves cutting off a body part, usually a limb or digit. Replantation involves reattaching a severed body part. Reconstructive surgery involves reconstruction of an injured, mutilated, or deformed part of the body. Cosmetic surgery is done to improve the appearance of an otherwise normal structure. Excision is the cutting out of an organ, tissue, or other body part from the patient. Transplant surgery is the replacement of an organ or body part by insertion of another from different human (or animal) into the patient. Removing an organ or body part from a live human or animal for use in transplant is also a type of surgery.

When surgery is performed on one organ system or structure, it may be classed by the organ, organ system or tissue involved. Examples include cardiac surgery (performed on the heart), gastrointestinal surgery (performed within the digestive tract and its accessory organs), and orthopedic surgery (performed on bones and/or muscles).

Minimally invasive surgery involves smaller outer incision(s) to insert miniaturized instruments within a body cavity or structure, as in laparoscopic surgery or angioplasty. By contrast, an open surgical procedure requires a large incision to access the area of interest. Laser surgery involves use of a laser for cutting tissue instead of a scalpel or similar surgical instruments. Microsurgery involves the use of an operating microscope for the surgeon to see small structures. Robotic surgery makes use of a surgical robot, such as Da Vinci or Zeus surgical systems, to control the instrumentation under the direction of the surgeon.

Advances in the field of surgery have allowed surgeons to perform procedures that were considered impossible 30 years ago, and similar improvements in operative management and surgical and imaging modalities, among many other factors, have paved the way for more aggressive approaches to surgical care. However, many of the operations performed every day carry significant risk, especially when blood flow must be altered to accommodate a surgical procedure.

Performing surgery on the liver, kidney, heart, brain, and other organs often requires that the surgical team stop blood flow to an organ or tissue to prevent massive bleeding and the possibility of terminal organ damage or exsanguination. Although the lack of blood and thus lack or oxygen may be tolerated by bodily tissue for variable durations, organs may suffer significant injury during the ischemic period, and especially during reperfusion following the surgical procedure. In light of the findings disclosed by the current inventors herein, it is proposed that γ-glutamylcysteine be used in the treatment of subjects undergoing a surgical procedure in order to protect against ischemia/reperfusion injury during any surgical procedure that involves alteration of blood flow to an organ or tissue in order to facilitate said surgical procedure.

C. Shock

Systemic shock is another situation encountered frequently by health care providers. Shock is defined as a physiologic state in which the body is unable to deliver sufficient oxygen to meet the demands of the body. Under normal conditions, oxygen delivery is five times higher than oxygen demand, creating a significant excess of oxygen. During shock, demand increases and supply decreases to the point that there is more oxygen consumption than delivery, resulting in hypoxia. Shock may be caused by cardiac insufficiency (cardiogenic shock, as in chronic heart failure, acute myocardial infarction, or following cardiac surgery), hypovolemia (as in hemorrhage, trauma, or dehydration), obstruction (as in pulmonary embolism or pericardial tamponade), sepsis, anaphylaxis, or spinal shock. Unintentional traumatic injury is an especially important cause of systemic shock, and is the number one killer of children and adolescents across the globe. Regardless of the etiology, systemic shock is characterized by low blood pressure and an inability of the circulatory system to deliver sufficient oxygen to meet the demands of the body. Unfortunately, by the time the circulatory system is stabilized, many patients have already sustained significant ischemic injury to their organs. The brain, kidneys, liver, bowel, and almost all other organs and tissues may be affected to some degree by such injury. The subsequent restoration of normal blood flow further subjects these organs and tissues to reperfusion injury, which can cause organ failure due to massive cell death.

The only way to prevent such injury is to not reperfuse, which is not compatible with life, and there is thus a need for efficacious drugs capable of ameliorating the ischemia/reperfusion injury in subjects experiencing systemic shock. It is proposed that γ-glutamylcysteine could be administered to a subject experiencing systemic shock in order to protect the subject's organs and tissues from ischemia/reperfusion injury.

### VI. MODES OF ADMINISTRATION AND PHARMACEUTICAL COMPOSITIONS

An effective amount of a γ-glutamylcysteine pharmaceutical composition, in the context of the present invention, is defined as that amount sufficient to improve the quality of transplanted material, or to reduce ischemia/reperfusion injury in tissues or organs with insufficient blood or oxygen delivery. More rigorous definitions may apply, including successful transplantation or protection of injured/damaged tissue from ischemic injury.

A. Exposure

The routes of administration of γ-glutamylcysteine will vary, naturally, with the cell type and tissue/organ location; however, generally cells/tissues/organs will be exposed to γ-glutamylcysteine by incubating them with γ-glutamylcysteine, immersing them in γ-glutamylcysteine, injecting them with γ-glutamylcysteine, or perfusing them with γ-glutamylcysteine.

Apparati discussed herein can be used to expose cells/tissues/organs to γ-glutamylcysteine. It is contemplated that γ-glutamylcysteine can be cycled in and out of a chamber or container in which the cells/tissues/organs are housed, or that the amount of γ-glutamylcysteine to which the cells are exposed can vary periodically or intermittently.

B. Formulations

Solutions of γ-glutamylcysteine may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms of γ-glutamylcysteine suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringeability exists. It must be stable under the conditions of manufacture and storage and must be preserved against the contaminating action of microorganisms, such as bacteria and fungi. The carrier can be a solvent or dispersion medium containing, for example, water, ethanol, DMSO, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by FDA Office of Biologics standards.

Sterile injectable solutions are prepared by incorporating the active compounds in the required amount in the appropriate solvent with various combinations of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” or “pharmacologically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

C. Perfusion Systems

A perfusion system for cells/tissues/organs may be used to expose materials to γ-glutamylcysteine in the form of a liquid or a semi-solid. Perfusion refers to continuous flow of a solution through or over a population of cells. It implies the retention of the cells within the culture unit as opposed to continuous-flow culture, which washes the cells out with the withdrawn media (e.g., chemostat). Perfusion allows for better control of the culture environment (pH, pO₂, nutrient levels, γ-glutamylcysteine, etc.) and is a means of significantly increasing the utilization of the surface area within a culture for cell attachment.

The technique of perfusion was developed to mimic the cells milieu in vivo where cells are continuously supplied with blood, lymph, or other body fluids. Without perfusion of a physiological nutrient solution, cells in culture go through alternating phases of being fed and starved, thus limiting full expression of their growth and metabolic potential. In the context of the present invention, a perfusion system may also be used to perfuse cells/tissues/organs with γ-glutamylcysteine.

Those of skill in the art are familiar with perfusion systems, and there are a number of perfusion systems available commercially. Any of these perfusion systems may be employed in the present invention. One example of a perfusion system is a perfused packed-bed reactor using a bed matrix of a non-woven fabric (CelliGen™, New Brunswick Scientific, Edison, N.J.; Wang et al., 1992; 1993; 1994). Briefly described, this reactor comprises an improved reactor for culturing of both anchorage- and non-anchorage-dependent cells. The reactor is designed as a packed bed with a means to provide internal recirculation. In one embodiment, a fiber matrix carrier is placed in a basket within the reactor vessel. A top and bottom portion of the basket has holes, allowing the medium to flow through the basket. A specially designed impeller provides recirculation of the medium through the space occupied by the fiber matrix for assuring a uniform supply of nutrient and the removal of wastes. This simultaneously assures that a negligible amount of the total cell mass is suspended in the medium. The combination of the basket and the recirculation also provides a bubble-free flow of oxygenated medium through the fiber matrix. The fiber matrix is a non-woven fabric having a “pore” diameter of from 10 μm to 100 μm, providing for a high internal volume with pore volumes corresponding to 1 to 20 times the volumes of individual cells.

The perfused packed-bed reactor offers several advantages. With a fiber matrix carrier, the cells are protected against mechanical stress from agitation and foaming. The free medium flow through the basket provides the cells with optimum regulated levels of oxygen, pH, and nutrients. Products can be continuously removed from the culture and the harvested products are free of cells and can be produced in low-protein medium, which facilitates subsequent purification steps. This technology is explained in detail in WO 94/17178 (Freedman et al.), which is hereby incorporated by reference in its entirety.

The Cellcube™ (Corning-Costar) module provides a large styrenic surface area for the immobilization and growth of substrate attached cells. It is an integrally encapsulated sterile single-use device that has a series of parallel culture plates joined to create thin sealed laminar flow spaces between adjacent plates.

The Cellcube™ module has inlet and outlet ports that are diagonally opposite each other and help regulate the flow of media. During the first few days of growth the culture is generally satisfied by the media contained within the system after initial seeding. The amount of time between the initial seeding and the start of the media perfusion is dependent on the density of cells in the seeding inoculum and the cell growth rate. The measurement of nutrient concentration in the circulating media is a good indicator of the status of the culture. When establishing a procedure it may be necessary to monitor the nutrient composition at a variety of different perfusion rates to determine the most economical and productive operating parameters.

Other commercially available perfusion systems include, for example, CellPerf® (Laboratories MABIO International, Tourcoing, France) and the Stovall Flow Cell (Stovall Life Science, Inc., Greensboro, N.C.).

### VII. COMBINATION TREATMENTS

The compounds and methods of the present invention may be used in the context of a number of therapeutic applications, particularly organ transplants or traumas. In order to increase the effectiveness of a treatment according to the present invention, γ-glutamylcysteine, it may be desirable to combine this treatment with other agents effective in the treatment of ischemia/reperfusion injury. For example, chemo-immunosuppressants may be administered in conjunction with transplantation. Moreover, additional drugs, such as those described in U.S. Pat. No. 6,552,083 (inhibitory agent comprising N-(3,4-dimethoxycinnamoyl)anthranililc acid) and U.S. Pat. No. 6,013,256 (antibodies that bind the IL-2 receptor, such as a humanized anti-Tax antibody), anti-oxidants or free radical scavengers may be employed.

Various combinations may be employed; for example, as shown below, γ-glutamylcysteine is “A” and the secondary therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of γ-glutamylcysteine of the present invention will follow general protocols for the administration of drugs and/or biologicals, taking into account the toxicity, if any, of the treatment. It is expected that the treatment cycles would be repeated as necessary.

### VIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### In Vitro Studies

The inventors have demonstrated that γ-glutamylcysteine (500 μM) is able to significantly reduce cell death after exposure to 10 μM H₂O₂. In addition, if neuronal cells are pretreated with GGC (500 μM) four hours prior to exposure to H₂O₂, the level of F₂-IsoPs, and therefore oxidative stress, is reduced. γ-glutamylcysteine has been shown in vitro to significantly reduce cellular death in response to an oxidative stress.

### Example 2

### In Vivo Studies

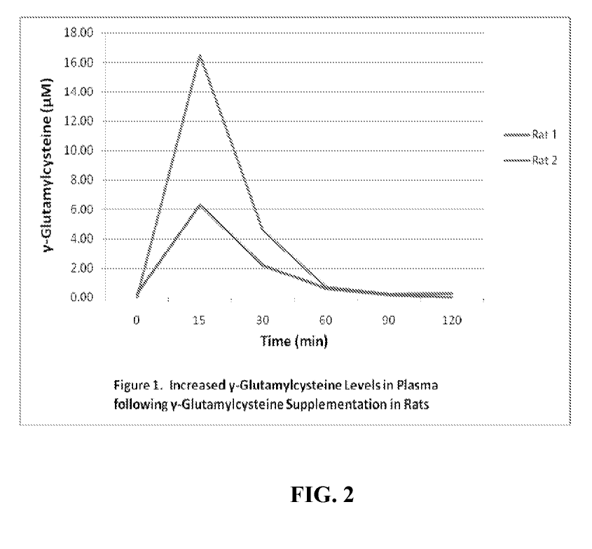

The inventors have demonstrated that intravenous γ-glutamylcysteine supplementation is well-tolerated in rats and results in significant elevation of plasma γ-glutamylcysteine levels as well as tissue glutathione levels. Two rats were administered γ-glutamylcysteine intravenously. Prior to sacrificing the animals, there were no clinically visible signs of toxicity in these animals at this dose. Plasma was collected at 0, 15, 30, 60, 90, and 120 minutes post-administration and assayed for γ-glutamylcysteine levels. As demonstrated in FIG. 2, both rats demonstrated increased plasma levels of γ-glutamylcysteine peak concentration at 30 minutes post-injection.

Ten rats were also administered 400 mg/kg of γ-glutamylcysteine intravenously. Major organ tissues were harvested from these rats at 0, 30, 60, 90, 120, and 240 minutes and assayed for glutathione concentration. As demonstrated in Table 1 (below), mean glutathione levels were increased in brain, heart, lung, liver, kidney, gastrointestinal, and muscle tissues with peak concentrations between 30 and 90 minutes.

TABLE 1
Tissue Glutathione Levels After Intravenous γ-Glutamylcysteine Supplementation

| | Glutathione Concentration (μM) | | | | |
|---|---|---|---|---|---|
| Time | 0 min | 60 min | 90 min | 120 min | 240 min |
| n | 5 | 4 | 2 | 2 | 2 |
| Brain | 57.3 (3.3-111.3) | 302.3 (190.0-414.5) | 146.9 (−1200.5-1494.3) | 170.7 (152.0-189.5) | 110.7 (54.1-167.3) |
| Heart | 210.9 (34.4-387.4) | 353.4 (220.9-486.0) | 658.9 (504.5-813.2) | 165.9 (149.4-182.4) | 278.1 (170.7-385.4) |
| Lung | 142.2 (50.8-233.7) | 293.5 (248.6-338.4) | 412.2 (−571.1-1395.4) | 166.4 (−58.3-391.0) | 93.2 (−261.7-448.1) |
| Liver | 2162.4 (1490.3-2834.5) | 5066.4 (−85.9-10218.8) | 10832.6 (9616.0-12049.1) | 2990.0 (2239.8-3740.1) | 4213.7 (−6431.5-14858.8) |
| Kidney | 14.9 (4.5-25.2) | 13.5 (−9.1-36.2) | 39.1 (−195.0-273.2) | 13.4 (−16.9-43.6) | 10.2 (−8.9-29.3) |
| GI | 12.9 (7.2-18.5) | 189.8 (−5.5-385.0) | 166.8 (−1699.0-2032.5) | 14.0 (−1.6-29.6) | 18.5 (−100.3-137.3) |
| Muscle | 228.5 (125-0-331.9) | 307.6 (−1.1-616.3) | 654.5 (44.8-1264.2) | 170.5 (−137.5-478.6) | 21.9 (−16.3-60.1) |

Values indicate mean tissue glutathione concentrations with 95% confidence interval in parentheses. n represents the number of animals used at each time point.

Pharmacokinetics of γ-Glutamylcysteine in Newborn Pigs. The inventors will perform a dose escalation of γ-glutamylcysteine in piglets with the goal to achieve concentrations of 100 μM, 200 μM, 500 μM and 1000 μM using three animals at each dose to monitor both blood and cerebrospinal fluid levels of the drug over a 4-hour period after a dose. The animals will be sedated, have a tracheostomy placed, and then be mechanically ventilated for this time period. Induction will be with ketamine and acepromazine, while anesthesia will be maintained with intermittent pentobarbital. This part of the study will involve 12 piglets.

Safety evaluation of γ-Glutamylcysteine in Newborn Pigs. Based on pharmacokinetic studies, the inventors will perform a safety evaluation by administering γ-glutamylcysteine intravenously to piglets to achieve three desired blood levels and monitoring the animals for seventy-two hours using vital signs, physical examination and laboratory (CBC, electrolytes, liver function) testing. This will be performed with 2 animals at each dose, and therefore involve 6 piglets.

Test γ-Glutamylcysteine in a Piglet Model of Hypoxia/Reperfusion Injury. If the compound is well tolerated in these animals, the inventors will test γ-glutamylcysteine in three doses (determined by previous pharmacokinetics) in a model of hypoxia-reperfusion injury as described by Cheung et al. using high dose epinephrine in hypoxic piglets resuscitated with 100% oxygen. Without any intervention in this model, Cheung et al. have shown a fall in cardiac index, stroke volume and an increase in arterial lactate and pulmonary vascular resistance that is mitigated by catecholamine administration. After exposure to an FIO₂ of 15% for 30 minutes, these animals develop pulmonary hypertension which this group previously has shown is responsive to IV sildenafil (Haase et al., Shock 26(1): 99-106). This model will closely mimic an infant's exposure to cardiopulmonary bypass.

After induction with ketamine and acepromazine, newborn piglets will have a tracheostomy performed and then be placed on a mechanical ventilator. Next, a femoral arterial catheter and aortic catheter will be placed. Finally, a thermodilution apparatus will be used with a catheter advanced into the pulmonary artery in order to measure pulmonary artery pressure as well as cardiac output. After these procedures, the piglet will be ventilated with approximately fifteen-percent inspired oxygen for 2 hours. Next, the piglet will be ventilated with 100% FIO₂ for one hour and then a FIO₂ of 21% for a final hour. Pentobarbital will be used to maintain adequate sedation during the procedures and monitoring period. Initially, these procedures will be performed in piglets without supplementation with γ-glutamylcysteine. Then, using three dosing regimens based on the pharmacokinetic and safety studies already performed, the inventors will intravenously dose these animals with γ-glutamylcysteine prior to exposing the animal to the four-hour study period. Laboratory testing will be performed at baseline and then hourly to include glutathione, glutathione disulfide, F₂IsoPs, isofurans, arterial lactate and pH. Pulmonary artery pressure will be monitored continuously, and cardiac output will be measured at baseline and then hourly. In order to detect a change in isoprostane/isofuran levels of greater than one standard deviation with a power of 80%, we plan to study 9 pairs of piglets in this part of the protocol.

In a clinical model in infants undergoing cardiopulmonary bypass for repair of congenital heart effects, determine the pharmacokinetics, safety profile and efficacy of γ-glutamylcysteine at preventing and increase in biochemical markers of oxidative stress including plasma isoprostanes and isofurans.

To determine the safety and pharmacokinetics of γ-Glutamylcysteine in infants. If γ-glutamylcysteine shows efficacy in reducing oxidative stress and/or pulmonary vascular resistance in the piglet model, safety and pharmacokinetic studies will be performed in infants admitted to the PCCU after repair of congenital heart disease. These studies will involve a dose escalation trial with approximately four patients at each dose and include repeated serum and urine sampling for a four-hour period after the dose. In addition, baseline, 4- and 24-hour serum samples will be collected to measure electrolytes, liver function and hematological indices for safety.

To determine efficacy of γ-Glutamylcysteine in prevention of oxidant injury and pulmonary hypertension in infants status post repair of congenital heart disease, particularly those infants at increased risk of developing pulmonary hypertension. If γ-Glutamylcysteine is found to be safe in infants, the inventors plan to test its efficacy in the prevention of oxidative injury after cardiopulmonary bypass. This will involve a blinded, placebo-controlled trial in infants status post repair of congenital heart disease using three doses determined from the pharmacokinetic studies and measuring primarily isoprostanes and isofurans as indicators of oxidative injury as well as end organ function including pulmonary reactivity, renal failure, days on ventilator and length of ICU stay as secondary outcomes.

To quantify the oxidative stress response, as well as modification of this response by γ-glutamylcysteine supplementation, the inventors will measure isoprostane and isofuran levels. Isoprostanes are prostaglandin like compounds that are produced without cyclooxygenase by free radical induced peroxidation of arachidonic acid. Prostaglandin F_{2α} like compounds, termed F₂-isoprostanes (F₂IsoPs) have been shown both in vitro and in vivo to be an accurate measure of oxidative stress. In the Biomarkers of Oxidative Stress Study (BOSS), a NIH sponsored trial, F₂-IsoPs were found to be the most sensitive and specific method to assess in vivo oxidative stress (Kadiiska et al., Free Radic Biol Med 38:698-710). In piglets, isoprostanes have been shown to cause concentration dependent contractions of the pulmonary arteries of newborn and 2 week old piglets (Gonzalez-Luiz et al., Pediatr Res 57(6):845-852).

Isofurans, substances also produced from the oxidation of arachidonic acid, are preferentially formed in settings of high oxygen tension (Fessel et al., Proc Natl Acad Sci USA 99(26):16713-8). It is therefore likely that in a model of hypoxic reperfusion injury it will be more accurate to measure levels of both isofurans and isoprostanes in order to determine the degree of oxidative stress. These can both be readily measured using gas chromatography mass spectrometry in plasma and urine. In addition, the inventors will measure levels of glutathione and glutathione disulfide in these animals both as an indicator of oxidative stress and the response to γ-glutamylcysteine therapy.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods, and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### IX. REFERENCES

The following references, to the extent that they provide exemplary procedural or other details supplementary to those set forth herein, are specifically incorporated herein by reference.
U.S. Pat. No. 3,777,507
U.S. Pat. No. 3,881,990
U.S. Pat. No. 3,995,444
U.S. Pat. No. 4,186,565
U.S. Pat. No. 4,292,817
U.S. Pat. No. 4,473,637
U.S. Pat. No. 4,502,295
U.S. Pat. No. 4,559,258
U.S. Pat. No. 4,723,974
U.S. Pat. No. 4,745,759
U.S. Pat. No. 4,798,824
U.S. Pat. No. 4,938,961
U.S. Pat. No. 4,951,482
U.S. Pat. No. 5,066,578
U.S. Pat. No. 5,157,930
U.S. Pat. No. 5,217,860
U.S. Pat. No. 5,285,657
U.S. Pat. No. 5,326,706
U.S. Pat. No. 5,370,989
U.S. Pat. No. 5,395,314
U.S. Pat. No. 5,405,742
U.S. Pat. No. 5,434,045
U.S. Pat. No. 5,466,468
U.S. Pat. No. 5,476,763
U.S. Pat. No. 5,552,267
U.S. Pat. No. 5,599,659
U.S. Pat. No. 5,693,462
U.S. Pat. No. 5,699,793
U.S. Pat. No. 5,719,174
U.S. Pat. No. 5,736,397
U.S. Pat. No. 5,752,929
U.S. Pat. No. 5,952,168
U.S. Pat. No. 6,013,256
U.S. Pat. No. 6,046,046
U.S. Pat. No. 6,054,261
U.S. Pat. No. 6,100,082
U.S. Pat. No. 6,187,529
U.S. Pat. No. 6,365,338
U.S. Pat. No. 6,490,880
U.S. Pat. No. 6,492,103
U.S. Pat. No. 6,524,785
U.S. Pat. No. 6,552,083
Bernard et al., J. Thorac. Cardiovasc. Surg., 90:235-242, 1985.
Chiba et al., Eur. J. Immunol., 26(5):1164-1169, 1996.
Fessel et al., Proc. Natl. Acad. Sci. USA., 99(26):16713-16718, 2002.
Gonzalez-Luis et al., Pediatr. Res., 57(6):845-852, 2005.
Hasse et al., Shock, 26(1):99-106, 2006.
Kadiiska et al., Free Radic. Biol. Med., 38(6):698-710, 2005.
Kuroda et al., Transplantation, 46(3):457-460, 1988.
Ledingham et al., Circulation, 82(Part 2)IV351-358, 1990.
Ledingham et al., J. Thorac. Cardiobasc. Surg., 93:240-246, 1987.
Menasche et al., Eur. J. Cardio. Thorax. Surg., 8:207-213, 1994.
PCT Appln. WO 94/17178
Remington's Pharmaceutical Sciences, 15^{th} Ed., pages 1035-1038 and 1570-1580, Mack Publishing Company, Easton, Pa., 1980.
Wang et al., Am. J. Physiol., 263(4 Pt 1):G480-486, 1992.
Wang et al., J. Physiol., 475(3):431-437, 1994.
Wang et al., Science, 259(5098):1173-1175, 1993.
Willis et al., Free Radic. Biol. Med., 34(1):72-76, 2003.

1. A method of preventing or ameliorating oxidative injury to a cell comprising contacting said cell with a composition comprising γ-glutamylcysteine (GGC).

2. The method of claim 1, wherein said cell is an endothelial cell, a brain cell such as a neuronal cell or an astrocyte cell, a muscle cell, a gastrointestinal tract cell, a cardiac cell, a lung cell, a liver cell, a kidney cell, or a skin cell.

3. The method of claim 1, wherein said GGC is provided to said cell at between about 10 and about 5000 μm.

4. The method of claim 1, wherein said GGC is provided to said cell at 10 μm, 25 μm, 50 μm, 100 μm, 150, μm, 200 μm, 250 μm, 300 μm, 350 μm, 400 μm, 450 μm, 500 μm, 600 μm, 700 μm, 800 μm, 900 μm, 1000 μm, 1500 μm, 2000 μm, 2500 μm, 3000 μm, 3500 μm, 4000 μm, 4500 μm, and 5000 μm.

5. The method of claim 1, further comprising contacting said cell with a free radical scavenger distinct from GGC.

6. A method of protecting a subject from oxidative injury comprising administering to said subject a pharmaceutical composition comprising γ-glutamylcysteine (GGC).

7. The method of claim 6, wherein said subject is a human, a dog, a cat, a pig, a horse, a cow, a rat, a mouse, a rabbit, a sheep, a goat, or a non-human primate.

8. The method of claim 6, wherein said composition is administered via intravenous, intra-arterial, subcutaneous, intramuscular, intraspinal, topical, dermal, intradermal, or intracardiac injection, oral delivery or via inhalation.

9. The method of claim 6, wherein said subject has or may be suffering from a stroke, a heart attack, pulmonary hypertension, hypoxia, trauma, chemical or toxin poisoning, or reperfusion injury.

10. The method of claim 9, wherein said composition is provided to said subject about 1, 2, 3, 4, 6, 8, 10 of 12 hours prior to and/or following the event.

11. The method of claim 6, wherein said subject is at risk of suffering from a stroke, a heart attack, pulmonary hypertension, hypoxia, chemical or toxin poisoning or reperfusion injury.

12. The method of claim 11, wherein said composition is provided to said subject about 1, 2, 3, 4, 6, 8, 10 of 12 hours prior to and/or following a risk-inducing event.

13. The method claim 6, further comprising administering said composition a second time.

14. The method of claim 6, wherein said composition comprises GGC at between about 10 and about 5000 μm.

15. The method of claim 6, wherein said composition comprises GGC at 10 μm, 25 μm, 50 μm, 100 μm, 150, μm, 200 μm, 250 μm, 300 μm, 350 μm, 400 μm, 450 μm, 500 μm, 600 μm, 700 μm, 800 μm, 900 μm, 1000 μm, 1500 μm, 2000 μm, 2500 μm, 3000 μm, 3500 μm, 4000 μm, 4500 μm, and 5000 μm.

16. The method of claim 6, further comprising administering to said subject a free radical scavenger distinct from GGC.
